(19) United States
(12) Patent Application Publication
(10) Pub. No.: US 2016/0186271 A1
(43) Pub. Date: Jun. 30, 2016

(54) COMPOSITIONS AND METHODS FOR DETERMINING THE PROGNOSIS OF BLADDER UROTHELIAL CANCER

(71) Applicant: ROSETTA GENOMICS LTD., Rehovot (IL)

(72) Inventors: Ofer Nativ, Haifa (IL); Yaron Goren, Kefar Hess (IL)

(21) Appl. No.: 14/976,978

(22) Filed: Dec. 21, 2015

Related U.S. Application Data
(63) Continuation of application No. 13/055,930, filed on Jan. 25, 2011, now abandoned.
(60) Provisional application No. 61/138,534, filed on Dec. 18, 2008.
(60) Provisional application No. 61/088,360, filed on Aug. 13, 2008.

(51) Int. Cl. C12Q 1/68

(52) U.S. Cl. CPC C12Q 1/6886; C12Q 2600/158; C12Q 2600/118; C12Q 2600/112; C12Q 2600/178

(57) ABSTRACT

Described herein are compositions and methods for the prediction of bladder cancer risk of invasiveness. The compositions are microRNA molecules associated with the prognosis of bladder cancer, as well as various nucleic acid molecules relating thereto or derived therefrom.

### CROSS REFERENCE TO RELATED APPLICATIONS

The present application claims priority under 35 U.S.C. §119(e) to U.S. Provisional Application No. 61/088,360, filed Aug. 13, 2008 and U.S. Provisional Application No. 61/138,534, filed Dec. 18, 2008 which are herein incorporated by reference in their entirety.

### FIELD OF THE INVENTION

The present invention relates to compositions and methods for the prediction of bladder cancer risk of invasiveness. Specifically the invention relates to microRNA molecules associated with the prognosis of bladder cancer, as well as various nucleic acid molecules relating thereto or derived thereof.

### BACKGROUND OF THE INVENTION

In recent years, microRNAs (miRs, miRNAs) have emerged as an important novel class of regulatory RNA, which have a profound impact on a wide array of biological processes. These small (typically 18-24 nucleotides long) non-coding RNA molecules can modulate protein expression patterns by promoting RNA degradation, inhibiting mRNA translation, and also affecting gene transcription. miRs play pivotal roles in diverse processes such as development and differentiation, control of cell proliferation, stress response and metabolism. The expression of many miRs was found to be altered in numerous types of human cancer, and in some cases strong evidence has been put forward in support of the conjecture that such alterations may play a causative role in tumor progression. There are currently about 800 known human miRs that regulate a postulated 30% or more of the human genes.

Urothelial carcinoma (UC) of the bladder is the fourth most common cancer in the western world, with estimated incidence of nearly 70,000 new cases and bladder cancer death over 14,000 in 2008 in the United States. At diagnosis, 70%-75% of bladder tumors are non muscle invasive tumors that do not invade into the smooth muscle fibers of the detrusor muscle. Approximately, 70% of these tumors are Ta, confined to the urothelium, 20% are T1, invade the lamina propria, and 10% are carcinoma in situ (CIS). Ta and T1, with their various grades, compose a heterogenous group of tumors with respect to prognosis. Low grade Ta lesions recur at a rate of 50%-70%, and progress to invasive cancer within 3 years in approximately 5% of the cases. on the other hand, high grade T1 tumors, recur in more than 80% of cases and in 50% of patients, progress within 3 years.

Recurrence and progression prediction is currently based upon clinical and pathological factors: tumor grade, tumor stage (T category), number of tumors, tumor size, prior recurrence rate, and presence of concomitant CIS. Tumor progression is affected mainly by the tumor grade and also by the T category and the presence of CIS, which are important risk factors. Low and high grade tumors present a vast gap in biological behavior and clinical outcome. High grade tumors display more evident chromosomal alterations and have a much poorer prognosis. The two kinds of bladder tumors are therefore often viewed as two different diseases. Despite the utilization of tumor grade and stage, along with the other predictors, the ability of these factors to assess patient prognosis is not satisfactory. Clinical behavior of urothelial cancer, especially high grade T1, is difficult to predict with present tools.

For example, in the current stratification, approximately 50% of patients diagnosed as high risk (high grade T1), in fact do not progress within 3 years, Since the follow-up and treatment regimes depend on prognosis, there is a need of more accurate stratification to increase the predictive values of risk groups. With a reliable diagnostic test for progression, suitable treatment could be tailored to every specific patient. Patients with tumors that would progress into muscle invasive disease would undergo an early radical cystectomy. Cure rates would be higher and unnecessary bothering and costly procedures would be prevented. Patients with tumors that would not invade the muscle layer could benefit a more convenient follow up, in terms of larger time interval between cystoscopies and operations, and avoiding unnecessary cystectomy, an operation with both significant morbidity and mortality.

This has led to an effort to find reliable biomarkers to predict progression of urothelial cancer. These potential markers include genetic alterations, cell adhesion molecules, proteases, growth factors and other molecular markers. To date, the markers that have been suggested lack sufficient predictive power for clinical evaluation of T1 urothelial cancer.

Although bladder cancer is often identified at an early stage, it is characterized by a high rate of recurrence with a risk for progression to invasive, fatal disease. Thus, patients are required to undergo frequent invasive follow-up procedures that are painful and costly, making bladder cancer a disproportionately heavy burden on health management. Reducing the frequency of follow-up can increase the fraction of cases where recurrent disease is only identified in an invasive stage. Prognostic markers that can accurately stratify patients into risk groups can aid in reducing both the burden of this disease and the disease-associated mortality, by identifying patients that require less frequent follow-up or more aggressive treatment.

At present, the most reliable way of diagnosis and surveillance of bladder cancer is cystoscopic examination and bladder biopsy for histological confirmation. The determination of the bladder cancer characteristics has a potential prognostic value and can be used to design an optimal therapy. Thus characterization of the molecular biological properties of a particular tumor could lead to a more specific and efficient therapy. According to the molecular basics of the tumor a follow-up protocol and a therapy could be tailored to avoid recurrence of the disease.

With a reliable diagnostic test for progression, suitable treatment could be tailored to every specific patient. Patients with tumors that would progress into muscle invasive disease would undergo an early radical cystectomy. Cure rates would be higher and unnecessary bothering and costly procedures would be prevented. Patients with tumors that would not invade the muscle layer could benefit a more convenient follow up, in terms of longer time interval between cystoscopies and operations, and avoiding unnecessary cystectomy, an operation with both significant morbidity and mortality.

Thus, there exists a need for identification of biomarkers that can be used as prognostic indicators for bladder cancer and for prediction the risk to develop invasive bladder disease.

### SUMMARY OF THE INVENTION

The present invention discloses for the first time the use of microRNA as a predictor of bladder tumor progression, in order to categorize and to distinguish between the different stages of bladder tumor in cancer.

According to the present invention altered expression levels of specific nucleic acid sequences (SEQ ID NOS: 8, 7, 1-6, 9-65) in biological samples obtained from bladder cancer patients is indicative of the cancer prognosis: the risk of invasiveness and the life expectancy of the patient.

According to one aspect of the invention, a method for determining a prognosis for bladder cancer in a subject is provided, the method comprising obtaining a biological sample from the subject, determining the expression level of a nucleic acid sequence selected from the group consisting of SEQ ID NOS: 8, 7, 1-6, 9-65 and sequences at least about 80% identical thereto from said sample; and comparing said expression level to a threshold expression level, wherein an altered expression level of the nucleic acid sequence compared to said threshold expression level is indicative of poor prognosis of said subject.

According to one embodiment, said altered expression level is an increased expression level and said nucleic acid sequence is selected from the group consisting of SEQ ID NOS: 1-6, 14, 16-21, 29, 31, 33, 34, 41-44, 48, 49, 51-53, 61-63 and sequences at least about 80% identical thereto.

According to another embodiment, said altered expression level is decreased expression level and said nucleic acid sequence is selected from the group consisting of SEQ ID NOS: 7-13, 15, 22-28, 30, 32, 35-40, 45-47, 50, 54-60, 64, 65 and sequences at least about 80% identical thereto.

According to yet another embodiment, said altered expression level is a change in a score based on a polynomial combination of expression level of said nucleic acid sequence.

In certain embodiments, said prognosis is prediction of bladder cancer risk of invasiveness.

According to another aspect of the invention, a method for distinguishing between stable non muscle invasive bladder cancer and unstable non muscle invasive bladder cancer is provided, the method comprising: obtaining a biological sample from a subject; determining in said sample an expression profile of nucleic acid sequences selected from the group consisting of SEQ ID NOS: 8, 7, 1-6, 9-65, a fragment thereof or a sequence having at least 80% identity thereto; and comparing said expression profile to a reference value; whereby a relative abundance of said nucleic acid sequences allows the detection of said bladder cancer.

According to some embodiments, a relative abundance of nucleic acid sequences selected from the group consisting of SEQ ID NOS: 1-6, 14, 16-21, 29, 31, 33, 34, 41-44, 48, 49, 51-53 and 61-63 and a sequence having at least 80% identity thereto is indicative of the presence of unstable non muscle invasive bladder cancer.

According to other embodiments, a relative abundance of nucleic acid sequences selected from the group consisting of SEQ ID NOS: 7-13, 15, 22-28, 30, 32, 35-40, 45-47, 50, 54-60, 64 and 65 and a sequence having at least 80% identity thereto is indicative of the presence of stable non muscle invasive bladder cancer.

In certain embodiments, the subject is a human.

In certain embodiments, the method is used to determine a course of treatment of the subject.

In certain embodiments the biological sample obtained from the subject is selected from the group consisting of bodily fluid, a cell line and a tissue sample. In certain embodiments the tissue is a fresh, frozen, fixed, wax-embedded or formalin fixed paraffin-embedded (FFPE) tissue.

In certain embodiments said tissue is a bladder tissue. In certain embodiments said tissue is a bladder non muscle invasive tumor tissue.

According to some embodiments, the expression levels are determined by a method selected from the group consisting of nucleic acid hybridization, nucleic acid amplification, and a combination thereof. According to some embodiments, the nucleic acid hybridization is performed using a solid-phase nucleic acid biochip array or in situ hybridization.

According to other embodiments, the nucleic acid amplification method is real-time PCR. According to some embodiments, the PCR method comprises forward and reverse primers. According to some embodiments the forward primers comprises a sequence selected from the group consisting of SEQ ID NOS: 66-70, a fragment thereof, and a sequence having at least about 80% identity thereto. According to some embodiments the reverse primer comprises SEQ ID NO: 76, a fragment thereof, and a sequence having at least about 80% identity thereto. According to some embodiments, the real-time PCR method further comprises a probe. According to some embodiments the probe comprises a sequence that is complementary to a sequence selected from the group consisting of SEQ ID NOS: 8, 7, 1-6, 9-65, a fragment thereof, and a sequence having at least about 80% identity thereto. According to some embodiments the probe comprises a sequence selected from the group consisting of SEQ ID NOS: 71-75, a fragment thereof, and a sequence having at least about 80% identity thereto.

A kit for determining the prognosis of a subject with bladder cancer is also provided. In some embodiments the kit comprises a probe comprising a nucleic acid sequence that is complementary to a sequence selected from the group consisting of SEQ ID NO: 8, 7, 1-6, 9-65; a fragment thereof and a sequence at least about 80% identical thereto. In some embodiments the probe comprises a nucleic acid sequence selected from SEQ ID NO: 71-75; a fragment thereof and a sequence at least about 80% identical thereto. According to other embodiments the kit further comprises forward and reverse primers. The forward primers may comprise a sequence selected from the group consisting of SEQ ID NOS: 66-70, a fragment thereof, and a sequence having at least about 80% identity thereto. The reverse primer may comprise SEQ ID NO: 76, a fragment thereof, and a sequence having at least about 80% identity thereto.

According to some embodiments, the kit comprises reagents for performing in situ hybridization analysis.

In some embodiments, prognostic for bladder cancer comprises providing the forecast or prediction of (prognostic for) any one or more of the following: risk of invasiveness, duration of survival of a patient susceptible to or diagnosed with bladder cancer, duration of recurrence-free survival, duration of progression free survival of a patient susceptible to or diagnosed with a cancer, response to treatment or response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. In some embodiments, duration of survival is forecast or predicted to be increased. In some embodiments duration of survival is forecast or predicted to be decreased. In some embodiments, duration of recurrence-free survival is forecast or predicted to be increased. In some embodiments duration of recurrence-free survival is forecast or predicted to be decreased. In some embodiments response rate is forecast or predicted to be increased. In some embodiments response rate is forecast or predicted to be decreased. In some embodiments, duration of response is predicted or forecast to be increased. In some embodiments, duration of response is predicted or forecast to be decreased. In some embodiments likelihood of metastasis is predicted or forecast to be increased. In some embodiments likelihood of metastasis is predicted or forecast to be decreased.

These and other embodiments of the present invention will become apparent in conjunction with the figures, description and claims that follow.

### BRIEF DESCRIPTION OF THE DRAWINGS

FIG. 1 is a scatter plot comparing the median expression levels of miRs (normalized fluorescence signals by microarray, shown in log-scale) in bladder tumor samples obtained from patients with invasive bladder cancer (T2 or more) (Y-axis, n=27) and patients with stable non muscle invasive tumors (no progression to invasive disease) (X-axis, n=26). The median values of each miR in all patients in one group were compared with the corresponding median for members of the other group. Each cross represents one miR. The parallel lines describe a fold change between groups of 1.5 in either direction. Statistically significant miRs are marked with circles: hsa-miR-21 (SEQ ID NO: 1), hsa-miR-150 (SEQ ID NO: 5), hsa-miR-146b-5p (SEQ ID NO: 2), hsa-miR-193a-3p (SEQ ID NO: 14), hsa-miR-18a (SEQ ID NO: 3), hsa-miR-31 (SEQ ID NO: 12), hsa-miR-29c (SEQ ID NO: 9), hsa-miR-10a (SEQ ID NO: 10), hsa-miR-26b (SEQ ID NO: 8), hsa-miR-29c* (SEQ ID NO: 15), hsa-miR-138 (SEQ ID NO: 7), hsa-miR-31* (SEQ ID NO: 11) and MID 00912 (SEQ ID NO: 4). P-values are calculated by two-sided unpaired student t-test, and significance is adjusted using FDR (false discovery rate) of 0.05. Not tested-control probes or median signal<300 in both groups.

FIG. 2 is a scatter plot comparing the median expression levels of miRs (normalized fluorescence signals by microarray, shown in log-scale) in bladder tumor samples obtained from patients with unstable non muscle invasive tumors (progression to invasive cancer was observed during follow-up, Y-axis, n=17) and patients with stable non muscle invasive tumors (X-axis, n=26). The median values of each miR in all patients in one group were compared with the corresponding median for members of the other group. Each cross represents one miR. The parallel lines describe a fold change between groups of 1.5 in either direction. Statistically significant miRs are marked with circles: hsa-miR-21 (SEQ ID NO: 1), hsa-miR-150 (SEQ ID NO: 5), hsa-miR-146b-5p (SEQ ID NO: 2), hsa-miR-193a-3p (SEQ ID NO: 14), hsa-miR-18a (SEQ ID NO: 3), hsa-miR-31 (SEQ ID NO: 12), hsa-miR-29c (SEQ ID NO: 9), hsa-miR-10a (SEQ ID NO: 10), hsa-miR-26b (SEQ ID NO: 8), hsa-miR-29c* (SEQ ID NO: 15), hsa-miR-138 (SEQ ID NO: 7), hsa-miR-31* (SEQ ID NO: 11) and MID 00912 (SEQ ID NO: 4). P-values are calculated by two sided Student t-test, and significance is adjusted using FDR (false discovery rate) of 0.05 and fold change 2.

FIG. 3 is a scatter plot comparing the median expression levels of miRs (normalized fluorescence signals by microarray, shown in log-scale) in bladder tumor samples obtained from patients with unstable non muscle invasive tumor (X-axis, n=17) and patients with invasive tumor (Y-axis, n=27). The median values of each miR in all patients in one group were compared with the corresponding median for members of the other group. Each cross represents one miR. The parallel lines describe a fold change between groups of 1.5 in either direction.

FIGS. 4A-4D are boxplot presentations comparing differences in the expression levels of the statistically significant miRs: hsa-miR-26b (SEQ ID NO: 8), (FIG. 4A); hsa-miR-138 (SEQ ID NO: 7), (FIG. 4B); hsa-miR-10a (SEQ ID NO: 10), (FIG. 4C); and hsa-miR-29c* (SEQ ID NO: 15), (FIG. 4D); in bladder tumor samples obtained from patients with stable non muscle invasive tumors that did not progress (left boxplot), patients with unstable non muscle invasive tumor that progressed (middle boxplot) or patients with invasive tumor (right boxplot). For each miR three boxes are shown respectively. The line in the box indicates the median value. The box top and bottom boundaries indicate the 25 and 75 percentile. The horizontal lines and crosses (outliers) show the full range of signals in this group. Units show log 2 of the normalized fluorescence signal.

FIG. 5A-5F are boxplot presentations comparing differences in the expression levels of the statistically significant miRs: hsa-miR-21 (SEQ ID NO: 1), (FIG. 5A); hsa-miR-193a-3p (SEQ ID NO: 14), (FIG. 5B); hsa-miR-18a (SEQ ID NO: 3), (FIG. 5C); hsa-miR-150 (SEQ ID NO: 5), (FIG. 5D); hsa-miR-125b (SEQ ID NO: 33), (FIG. 5E); and hsa-miR-25 (SEQ ID NO: 42), (FIG. 5F); in bladder tumor samples obtained from patients with stable non muscle invasive tumors (left boxplot), patients with unstable non muscle invasive tumors (middle boxplot) and patients with invasive tumor (right boxplot). For each miR three boxes are shown respectively. The line in the box indicates the median value. The box top and bottom boundaries indicate the 25 and 75 percentile. The horizontal lines and crosses (outliers) show the full range of signals in this group. Units show log 2 of the normalized fluorescence signal.

FIGS. 6A and 6B demonstrate the classification of bladder tumors using the expression levels of two microRNA biomarkers that have different expression levels in stable non muscle invasive tumors (diamond symbols), unstable non muscle invasive tumors (square symbols) and invasive tumors (circle symbols). The diagonal line represents a possible binary classification such that patients below it may be treated aggressively.

FIG. 6A shows the expression levels of hsa-miR-26b (SEQ ID NO: 8, Y-axis) and hsa-miR-193a-3p (SEQ ID NO: 14, X-axis).

FIG. 6B shows the expression levels of hsa-miR-26b (SEQ ID NO: 8, Y-axis) and hsa-miR-125 b (SEQ ID NO: 33, X-axis).

FIGS. 7A and 7B demonstrate the classification of bladder tumors using the expression levels of hsa-miR-26b (SEQ ID NO: 8), which is downregulated in invasive tumors.

FIG. 7A shows the expression levels of hsa-miR-26b (SEQ ID NO: 8, Y-axis) for each of the 26 stable non muscle invasive tumors that did not progress (circles), 18 unstable non muscle invasive tumors that progressed (diamonds), and 29 invasive bladder tumors (dark squares). The horizontal line shows a cutoff at hsa-miR-26b=3020 which has sensitivity of 100% (18 of 18) and specificity of 88% (23 of 26) for identifying non muscle invasive tumors that will become invasive (IP). The expression level of hsa-miR-26b has an AUC of 0.92 for separating the two types of non muscle invasive bladder tumors (IP vs. NP).

FIG. 7B is a Kaplan-Meier plot showing the progression-free survival (Y-axis) based on expression of hsa-miR-26b (SEQ ID NO: 8). Data is shown for the 26 non muscle invasive cases that did not progress (NP), and for 11 of the 18 non muscle invasive cases that progressed (IP) for whom detailed follow-up information was available including time to progression (months, X-axis). The 37 cases are divided according to the expression of hsa-miR-26b, into 23 individuals whose non muscle invasive tumors had a high expression level of hsa-miR-26b (solid line), and 14 individuals whose non muscle invasive tumors had a low expression level of hsa-miR-26b (dashed line). The group with high expression of hsa-miR-26b had no cases of tumor progression (FIG. 7A). The group with low expression of hsa-miR-26b had a median progression-free survival of 5 months. The difference in progression-free survival was highly significant (p-value 4.3e-7 by log rank test).

FIGS. 8A and 8B are scatter plots showing that the expression levels of hsa-miR-26 b (SEQ ID NO: 8, X-axis) and hsa-miR-138 (SEQ ID NO: 7, Y-axis) in bladder tumor samples obtained from patients with stable non muscle invasive tumors (circles), and in bladder tumor samples obtained from patients with unstable non muscle invasive tumors (diamonds), can be used to classify non muscle invasive bladder tumors into non muscle invasive cases that progressed (white gray area), stable non muscle invasive tumors that did not progress (dark area) and undetermined (light gray area). FIGS. 8A and 8B show the reproducibility of the results on PCR platform.

FIG. 8A presents the expression results of the microRNA array (normalized fluorescence signals, shown in log-scale) on hsa-miR-26b (X-axis) and hsa-miR-138 (Y-axis). A subset of these samples was chosen for validation on PCR platform.

FIG. 8B presents the expression results (as 50-Ct) of the RT-PCR assay on the samples selected for validation on the same microRNAs (hsa-miR-26b on the X-axis and hsa-miR-138 on the Y-axis). The discrimination power of these two microRNAs is similar when using RT-PCR and the same sample which was misclassified on the microRNA array (marked with a black dot) was also misclassified when using RT-PCR.

### DETAILED DESCRIPTION

According to the present invention miRNA expression can serve as a tool for the prediction of bladder cancer risk of invasiveness. More particularly, it may serve for distinguishing between stable non muscle invasive bladder cancer (which does not progress to invasiveness) and unstable non muscle invasive bladder cancer (which does progress to invasiveness). Methods and compositions are provided for the prognosis of bladder cancer.

In the present invention, determining the presence of said microRNA levels in biopsies, tumor samples, cells, tissues or bodily fluid, is particularly useful for discriminating between different subtypes of bladder tumors.

All the methods of the present invention may optionally further include measuring levels of other cancer markers. Other cancer markers, in addition to said microRNA molecules, useful in the present invention will depend on the cancer being tested and are known to those of skill in the art.

Assay techniques that can be used to determine levels of gene expression, such as the nucleic acid sequence of the present invention, in a sample derived from a patient are well known to those of skill in the art. Such assay methods include, but are not limited to, radioimmunoassays, reverse transcriptase PCR (RT-PCR) assays, immunohistochemistry assays, in situ hybridization assays, competitive-binding assays, Northern Blot analyses, ELISA assays, nucleic acid microarrays and biochip analysis.

An arbitrary threshold on the expression level of one or more nucleic acid sequences can be set for assigning a sample or tumor sample to one of two groups. Alternatively, in a preferred embodiment, expression levels of one or more nucleic acid sequences of the invention are combined by a method such as logistic regression to define a metric which is then compared to previously measured samples or to a threshold. The threshold for assignment is treated as a parameter, which can be used to quantify the confidence with which samples are assigned to each class. The threshold for assignment can be scaled to favor sensitivity or specificity, depending on the clinical scenario. The correlation value to the reference data generates a continuous score that can be scaled and provides diagnostic information on the likelihood that a sample belongs to a certain class of bladder carcinoma subtype. In multivariate analysis, the microRNA signature provides a high level of prognostic information.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

a. Definitions

Attached

“Attached” or “immobilized” as used herein to refer to a probe and a solid support may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

Biological Sample

“Biological sample” as used herein may mean a sample of biological tissue or fluid that comprises nucleic acids. Such samples include, but are not limited to, tissue isolated from animals. Biological samples may also include sections of tissues such as biopsy and autopsy samples, frozen sections taken for histological purposes, blood, plasma, serum, sputum, stool, tears, mucus, urine, effusions, amniotic fluid, ascitic fluid, hair, and skin. Biological samples also include explants and primary and/or transformed cell cultures derived from patient tissues. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

Cancer Prognosis

A forecast or prediction of the probable course or outcome of the cancer. As used herein, cancer prognosis includes the forecast or prediction of any one or more of the following: prediction of cancer risk of invasiveness, duration of survival of a patient susceptible to or diagnosed with a cancer, duration of recurrence-free survival, duration of progression free survival of a patient susceptible to or diagnosed with a cancer, response to treatment (such as chemotherapy, radiation, immunotherapy or any combination thereof) or response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: prediction of cancer risk of invasiveness, duration of survival of a patient susceptible to or diagnosed with a cancer, duration of recurrence-free survival, duration of progression free survival of a patient susceptible to or diagnosed with a cancer, response to treatment or response rate in a group of patients susceptible to or diagnosed with a cancer, duration of response to any method used for treatment of the condition in a patient or a group of patients susceptible to or diagnosed with a cancer, and/or likelihood of metastasis in a patient susceptible to or diagnosed with a cancer.

Classification

The term classification refers to a procedure and/or algorithm in which individual items are placed into groups or classes based on quantitative information on one or more characteristics inherent in the items (referred to as traits, variables, characters, features, etc) and based on a statistical model and/or a training set of previously labeled items.

Complement

“Complement” or “complementary” as used herein to refer to a nucleic acid may mean Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

C_{T}

C_{T} signals represent the first cycle of PCR where amplification crosses a threshold (cycle threshold) of fluorescence. Accordingly, low values of C_{T} represent high abundance or expression levels of the microRNA.

In some embodiments the PCR C_{T} signal is normalized such that the normalized C_{T} remains inversed from the expression level. In other embodiments the PCR C_{T} signal may be normalized and then inverted such that low normalized-inverted C_{T} represents low abundance or expression levels of the microRNA.

Differential Expression

“Differential expression” may mean qualitative or quantitative differences in the temporal and/or cellular gene expression patterns within and among cells and tissue. Thus, a differentially expressed gene can qualitatively have its expression altered, including an activation or inactivation, in, e.g., normal versus disease tissue. Genes may be turned on or turned off in a particular state, relative to another state thus permitting comparison of two or more states. A qualitatively regulated gene will exhibit an expression pattern within a state or cell type that may be detectable by standard techniques. Some genes will be expressed in one state or cell type, but not in both. Alternatively, the difference in expression may be quantitative, e.g., in that expression is modulated, up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, Northern analysis, and RNase protection.

Expression Profile

“Expression profile” as used herein may mean a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cRNA, etc., quantitative PCR, ELISA for quantification, and the like, and allow the analysis of differential gene expression between two samples. A subject or patient tumor sample, e.g., cells or collections thereof, e.g., tissues, is assayed. Samples are collected by any convenient method, as known in the art. Nucleic acid sequences of interest are nucleic acid sequences that are found to be predictive, including the nucleic acid sequences provided above, where the expression profile may include expression data for 5, 10, 20, 25, 50, 100 or more of, including all of the listed nucleic acid sequences. The term “expression profile” may also mean measuring the abundance of the nucleic acid sequences in the measured samples.

Expression Ratio

“Expression ratio” as used herein refers to relative expression levels of two or more nucleic acids as determined by detecting the relative expression levels of the corresponding nucleic acids in a biological sample.

FDR

When performing multiple statistical tests, for example in comparing the signal between two groups in multiple data features, there is an increasingly high probability of obtaining false positive results, by random differences between the groups that can reach levels that would otherwise be considered as statistically significant. In order to limit the proportion of such false discoveries, statistical significance is defined only for data features in which the differences reached a p-value (such as by a two-sided t-test) below a threshold, which is dependent on the number of tests performed and the distribution of p-values obtained in these tests. FDR or false discovery rate is the probability that one of the “significant” results was actually false.

Gene

“Gene” used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be a mRNA or cDNA corresponding to the coding regions (e.g., exons and miRNA) optionally comprising 5′- or Y-untranslated sequences linked thereto. A gene may also be an amplified nucleic acid molecule produced in vitro comprising all or a part of the coding region and/or 5′- or 3′-untranslated sequences linked thereto.

Identity

“Identical” or “identity” as used herein in the context of two or more nucleic acids or polypeptide sequences may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

Label

“Label” as used herein may mean a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

Logistic Regression

Logistic regression is part of a category of statistical models called generalized linear models. Logistic regression allows one to predict a discrete outcome, such as group membership, from a set of variables that may be continuous, discrete, dichotomous, or a mix of any of these. The dependent or response variable is dichotomous, for example, one of two possible types of cancer. Logistic regression models the natural log of the odds ratio, i.e. the ratio of the probability of belonging to the first group (P) over the probability of belonging to the second group (1-P), as a linear combination of the different expression levels (in log-space) and of other explaining variables. The logistic regression output can be used as a classifier by prescribing that a case or sample will be classified into the first type if P is greater than 0.5 or 50%. Alternatively, the calculated probability P can be used as a variable in other contexts such as a 1D or 2D threshold classifier.

1D/2D Threshold Classifier

“1D/2D threshold classifier” used herein may mean an algorithm for classifying a case or sample such as a cancer sample into one of two possible types such as two types of cancer or two types of prognosis (e.g. good and bad). For a 1D threshold classifier, the decision is based on one variable and one predetermined threshold value; the sample is assigned to one class if the variable exceeds the threshold and to the other class if the variable is less than the threshold. A 2D threshold classifier is an algorithm for classifying into one of two types based on the values of two variables. A score may be calculated as a function (usually a continuous function) of the two variables; the decision is then reached by comparing the score to the predetermined threshold, similar to the 1D threshold classifier.

Mismatch

“Mismatch” means a nucleobase of a first nucleic acid that is not capable of pairing with a nucleobase at a corresponding position of a second nucleic acid.

Nucleic Acid

“Nucleic acid” or “oligonucleotide” or “polynucleotide” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005), Soutschek et al., Nature 432:173-178 (2004), and U.S. Patent Publication No. 20050107325, which are incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver and kidney. Mixtures of naturally occurring nucleic acids and analogs may be made; alternatively, mixtures of different nucleic acid analogs, and mixtures of naturally occurring nucleic acids and analogs may be made.

Probe

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

Reference Value

As used herein the term “reference value” means a value that statistically correlates to a particular outcome when compared to an assay result. In preferred embodiments the reference value is determined from statistical analysis of studies that compare microRNA expression with known clinical outcomes.

Sensitivity

“sensitivity” used herein may mean a statistical measure of how well a binary classification test correctly identifies a condition, for example how frequently it correctly classifies a cancer into the correct type out of two possible types. The sensitivity for class A is the proportion of cases that are determined to belong to class “A” by the test out of the cases that are in class “A”, as determined by some absolute or gold standard.

Specificity

“Specificity” used herein may mean a statistical measure of how well a binary classification test correctly identifies a condition, for example how frequently it correctly classifies a cancer into the correct type out of two possible types. The specificity for class A is the proportion of cases that are determined to belong to class “not A” by the test out of the cases that are in class “not A”, as determined by some absolute or gold standard.

Stable Non Muscle Invasive Tumor

A tumor which does not progress to an invasive disease. As used herein a non-invasive tumor sample was classified stable non muscle invasive if no progression occurred within 5 years.

Stage of Cancer

As used herein, the term “stage of cancer” refers to a numerical measurement of the level of advancement of a cancer. Criteria used to determine the stage of a cancer include, but are not limited to, the degree of invasion of the various layers of the bladder wall, invasion of lymph and blood vessels, involvement of perivesical structures, regional or systemic lymph nodes and whether the tumor has spread to other parts of the body.

Stringent Hybridization Conditions

“Stringent hybridization conditions” used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

Substantially Complementary

“Substantially complementary” used herein may mean that a first sequence is at least 60%-99% identical to the complement of a second sequence over a region of 8-50 or more nucleotides, or that the two sequences hybridize under stringent hybridization conditions.

Substantially Identical

“Substantially identical” used herein may mean that a first and second sequence are at least 60%-99% identical over a region of 8-50 or more nucleotides or amino acids, or with respect to nucleic acids, if the first sequence is substantially complementary to the complement of the second sequence.

Subject

As used herein, the term “subject” refers to a mammal, including both human and other mammals. The methods of the present invention are preferably applied to human subjects.

Therapeutically Effective Amount

As used herein the term “therapeutically effective amount” or “therapeutically efficient” as to a drug dosage, refer to dosage that provides the specific pharmacological response for which the drug is administered in a significant number of subjects in need of such treatment. The “therapeutically effective amount” may vary according, for example, the physical condition of the patient, the age of the patient and the severity of the disease. Radiotherapy may also be given or combination treatment.

Threshold Expression Level

As used herein, the phrase “threshold expression level” refers to a criterion expression value to which measured values are compared in order to determine the prognosis of a subject with bladder cancer. Typically a reference threshold expression value will be a threshold above which one outcome is more probable and below which an alternative threshold is more probable.

Treat

“Treat” or “treating” used herein when referring to protection of a subject from a condition may mean preventing, suppressing, repressing, or eliminating the condition. Preventing the condition involves administering a composition described herein to a subject prior to onset of the condition. Suppressing the condition involves administering the composition to a subject after induction of the condition but before its clinical appearance. Repressing the condition involves administering the composition to a subject after clinical appearance of the condition such that the condition is reduced or prevented from worsening. Elimination of the condition involves administering the composition to a subject after clinical appearance of the condition such that the subject no longer suffers from the condition.

Tumor

“Tumor” as used herein, refers to all neoplastic cell growth and proliferation, whether malignant or benign, and all pre-cancerous and cancerous cells and tissues.

Unstable Non Muscle Invasive Tumor

A tumor which progresses to an invasive disease. As used herein a non-invasive tumor sample was classified unstable non muscle invasive if progression occurred within 5 years.

Variant

“Variant” used herein to refer to a nucleic acid may mean (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

b. MicroRNA and its Processing

A gene coding for a miRNA may be transcribed leading to production of a miRNA precursor known as the pri-miRNA. The pri-miRNA may be part of a polycistronic RNA comprising multiple pri-miRNAs. The pri-miRNA may form a hairpin with a stem and loop. The stem may comprise mismatched bases.

The hairpin structure of the pri-miRNA may be recognized by Drosha, which is an RNase III endonuclease. Drosha may recognize terminal loops in the pri-miRNA and cleave approximately two helical turns into the stem to produce a 30-200 nt precursor known as the pre-miRNA. Drosha may cleave the pri-miRNA with a staggered cut typical of RNase III endonucleases yielding a pre-miRNA stem loop with a 5′ phosphate and nucleotide 3′ overhang. Approximately one helical turn of stem (˜10 nucleotides) extending beyond the Drosha cleavage site may be essential for efficient processing. The pre-miRNA may then be actively transported from the nucleus to the cytoplasm by Ran-GTP and the export receptor Ex-portin-5.

The pre-miRNA may be recognized by Dicer, which is also an RNase III endonuclease. Dicer may recognize the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specifity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miRNA and the mRNA, especially by nucleotides 2-8 of the miRNA. Only one case has been reported in animals where the interaction between the miRNA and its target was along the entire length of the miRNA. This was shown for miR-196 and Hox B8 and it was further shown that miR-196 mediates the cleavage of the Hox B8 mRNA (Yekta et at 2004, Science 304-594). Otherwise, such interactions are known only in plants (Bartel & Bartel 2003, Plant Physiol 132-709).

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5° UTR, the 3 UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

c. Nucleic Acids

Nucleic acids are provided herein. The nucleic acid may comprise the sequence of SEQ ID NOS: 1-76 presented in tables 1 and 2 or variants thereof. The variant may be a complement of the referenced nucleotide sequence. The variant may also be a nucleotide sequence that is substantially identical to the referenced nucleotide sequence or the complement thereof. The variant may also be a nucleotide sequence which hybridizes under stringent conditions to the referenced nucleotide sequence, complements thereof, or nucleotide sequences substantially identical thereto.

The nucleic acid may have a length of from 10 to 250 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200 or 250 nucleotides. The nucleic acid may be synthesized or expressed in a cell (in vitro or in vivo) using a synthetic gene described herein. The nucleic acid may be synthesized as a single strand molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated by reference.

i. Nucleic Acid Complex

The nucleic acid may further comprise one or more of the following: a peptide, a protein, a RNA-DNA hybrid, an antibody, an antibody fragment, a Fab fragment, and an aptamer. The nucleic acid may also comprise a protamine-antibody fusion protein as described in Song et at (Nature Biotechnology 2005; 23:709-17) and Rossi (Nature Biotechnology 2005:23; 682-4), the contents of which are incorporated herein by reference. The protamine-fusion protein may comprise the abundant and highly basic cellular protein protamine. The protamine may readily interact with the nucleic acid. The protamine may comprise the entire 51 amino acid prolamine peptide or a fragment thereof. The protamine may be covalently attached to another protein, which may be a Fab. The Fab may bind to a receptor expressed on a cell surface.

ii. Pri-miRNA

The nucleic acid may comprise a sequence of a pri-miRNA or a variant thereof. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof. The sequence of the pri-miRNA may comprise the sequence of SEQ ID NOS: 1-65 or variants thereof.

The pri-miRNA may form a hairpin structure. The hairpin may comprise first and second nucleic acid sequence that are substantially complimentary. The first and second nucleic acid sequence may be from 37-50 nucleotides. The first and second nucleic acid sequence may be separated by a third sequence of from 8-12 nucleotides. The hairpin structure may have a free energy less than −25 Kcal/mole as calculated by the Vienna algorithm with default parameters, as described in Hofacker et al., Monatshefie f. Chemie 125: 167-188 (1994), the contents of which are incorporated herein. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

iii. Pre-miRNA

The nucleic acid may also comprise a sequence of a pre-miRNA or a variant thereof. The pre-miRNA sequence may comprise from 45-200, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-65 or variants thereof.

iv. MiRNA

The nucleic acid may also comprise a sequence of a miRNA (including miRNA*) or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence derived from SEQ ID NOS: 1-65, or variants thereof.

v. Anti-miRNA

The nucleic acid may also comprise a sequence of an anti-miRNA that is capable of blocking the activity of a miRNA or miRNA*, such as by binding to the pri-miRNA, pre-miRNA, miRNA or miRNA.* (e.g. antisense or RNA silencing), or by binding to the target binding site. The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the anti-miRNA may comprise (a) at least 5 nucleotides that are substantially identical or complimentary to the 5′ of a miRNA and at least 5-12 nucleotides that are substantially complimentary to the flanking regions of the target site from the 5′ end of the miRNA, or (b) at least 5-12 nucleotides that are substantially identical or complimentary to the 3′ of a miRNA and at least 5 nucleotide that are substantially complimentary to the flanking region of the target site from the 3′ end of the miRNA. The sequence of the anti-miRNA may comprise the compliment of SEQ ID NOS: 1-65, or variants thereof.

vi. Binding Site of Target

The nucleic acid may also comprise a sequence of a target miRNA binding site, or a variant thereof. The target site sequence may comprise a total of 5-100 or 10-60 nucleotides. The target site sequence may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 nucleotides. The target site sequence may comprise at least 5 nucleotides of the complementarity sequence of SEQ ID NOS: 1-65.

d. Synthetic Gene

A synthetic gene is also provided comprising a nucleic acid described herein operably linked to a transcriptional and/or translational regulatory sequence. The synthetic gene may be capable of modifying the expression of a target gene with a binding site for a nucleic acid described herein. Expression of the target gene may be modified in a cell, tissue or organ. The synthetic gene may be synthesized or derived from naturally-occurring genes by standard recombinant techniques. The synthetic gene may also comprise terminators at the 3′-end of the transcriptional unit of the synthetic gene sequence. The synthetic gene may also comprise a selectable marker.

e. Probes

A probe is also provided comprising a nucleic acid described herein. Probes may be used for screening and diagnostic methods, as outlined below. The probe may be attached or immobilized to a solid substrate, such as a biochip.

The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may further comprise a linker sequence of from 10-60 nucleotides.

f. Biochip

A biochip is also provided. The biochip may comprise a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

g. Diagnosis

A method of diagnosis is also provided. The method, comprises detecting a differential expression level of bladder cancer-associated nucleic acid in a biological sample. The sample may be derived from a patient. Diagnosis of a disease state in a patient may allow for prognosis, selection of therapeutic strategy and follow-up strategy. Furthermore, the developmental stage of cells may be classified by determining temporarily expressed bladder cancer-associated nucleic acids.

In situ hybridization of labeled probes to tissue sections may be performed. When comparing the fingerprints between an individual and a standard, the skilled artisan can make a diagnosis, a prognosis, or a prediction of tumor invasiveness based on the findings. It is further understood that the nucleic acids which indicate the diagnosis may differ from those which indicate the prognosis and molecular profiling of the condition of the cells may lead to distinctions between responsive or refractory conditions or may be predictive of outcomes.

h. Biomarkers

Biomarkers are also provided. One type of cancer screening test involves the detection of a biomarker, such as a tumor marker, in a fluid or tissue obtained from a patient. Another important use for tumor markers is for monitoring patients being treated for advanced cancer. Measuring tumor markers for this purpose can be less invasive, less time-consuming, than other complicated tests, to determine if a therapy is reducing the cancer.

A further important use for tumor markers is for determining a prognosis of survival of a cancer patient. Such prognostic methods can be used to identify surgically treated patients likely to experience cancer invasiveness or recurrence so that they can be offered additional therapeutic options. Biomarkers useful for prognosis of survival also can be especially effective for determining the risk of metastasis in patients who demonstrate no measurable metastasis at the time of examination or surgery. Knowledge of the likelihood of metastasis in a cancer patient can be an important factor in selecting a treatment option. For example, a cancer patient likely to experience metastasis may be advantageously treated using a modality that is particularly aggressive.

i. Kits

A kit is also provided and may comprise a nucleic acid described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

For example, the kit may be a kit for the amplification, detection, identification or quantification of a target nucleic acid sequence. The kit may comprise a poly(T) primer, a forward primer, a reverse primer, and a probe.

Having now generally described the invention, the same will be more readily understood through reference to the following examples, which are provided by way of illustration and are not intended to be limiting of the present invention.

### EXAMPLES

### Example 1

### Materials and Methods

a. Biological Samples

73 primary bladder tumor specimens (formalin fixed, paraffin-embedded, FFPE) obtained by bladder cytoscopy and transurethral resection procedure were included in the study. This study was undertaken with the approval of the internal review boards of Soroka University Medical Center.

Total RNA enriched in microRNA was isolated from the FFPE bladder tumor specimens and all RNAs extracted were hybridized onto microarrays according to the RNA extraction and miR array platform protocols described below.

Of the 73 samples, cohort sizes were:

Stable non muscle invasive (the sampled tumor was non-invasive and no progression occurred within 5 years)-n=26

Unstable non muscle invasive (the sampled tumor was non-invasive and progression occurred within 5 years)-n=18

Invasive (the sampled tumor was invasive)-n=29

b. RNA Extraction

Total RNA was isolated from seven to ten 10-μm-thick FFPE tissue sections per case using the extraction protocol developed at Rosetta Genomics. Briefly, the sample was incubated a few times in xylene at 57° to remove excess paraffin, followed by Ethanol washes. Proteins were degraded by incubating the sample in a proteinase K solution at 45° C. for few hours. The RNA was extracted using acid phenol/chloroform followed by ethanol precipitation and DNAse digestion. Total RNA quantity and quality was measured by Nanodrop ND-1000 (NanoDrop Technologies, Wilmington, Del.).

c. Microarray

Custom microRNA microarrays were prepared by printing DNA oligonucleotide probes representing 688 human microRNAs. Each probe, printed in triplicate, carries up to 22-nt linker at the 3′ end of the microRNA's complement sequence in addition to an amine group used to couple the probes to coated glass slides. 20 μM of each probe were dissolved in 2×SSC 0.0035% SDS and spotted in triplicate on Schott Nexterion® Slide E coated microarray slides using a Genomic Solutions® BioRoboties MicroGrid II according the MicroGrid manufacturer's directions. 54 negative control probes were designed using the sense sequences of different microRNAs. Two types of positive control probes were included in the experimental design (i) synthetic small RNAs were spiked into each RNA sample before labeling to verify labeling efficiency and (ii) probes for abundant small RNAs (e.g. small nuclear RNAs (U43, U49, U24, Z30, U6, U48, U44), 5.8s and 5s ribosomal RNA) were spotted on the array to validate RNA quality. The slides were blocked in a solution containing 50 mM ethanolamine, 1M Tris (pH9.0) and 0.1% SDS for 20 min at 50° C., then thoroughly rinsed with water and spun dry.

d. Cy-Dye Labeling of microRNA for miR Array

3.5 μg of total RNA were labeled by ligation of an RNA-linker, p-rCrU-Cy/dye (Dharmacon, Lafayette, Colo.; Cy3 or Cy5) to the 3′ end. The labeling reaction contained total RNA, spikes (0.1-20 fmoles), 300 ng RNA-linker-dye, 15% DMSO, 1× ligase buffer and 20 units of T4 RNA ligase (NEB) and proceeded at 4° C. for 1 hr followed by 1 hr at 37° C. The labeled RNA was mixed with 3× hybridization buffer (Ambion), heated to 95° C. for 3 min and then added on top of the miR array. Slides were hybridized 12-16 hr in 42° C., followed by two washes in room temperature with 1×SSC and 0.2% SDS and a final wash with 0.1×SSC.

Arrays were scanned using an Agilent Microarray Scanner Bundle G2565BA (resolution of 10 μm at 100% power). Array images were analyzed using SpotReader software (Niles Scientific, Portola Valley, Calif.).

e. Array Data Normalization

The initial data set consisted of signals measured for multiple probes for every sample. For the analysis, signals were used only for probes that were designed to measure the expression levels of known or validated human microRNAs.

Triplicate spots were combined into one signal by taking the logarithmic mean of the reliable spots. All data was log-transformed and the analysis was performed in log-space. A reference data vector for normalization, R, was calculated by taking the median expression level for each probe across all samples.

For each sample k with data vector S^{k}, a 2nd degree polynomial F^{k} was found so as to provide the best fit between the sample data and the reference data, such that R≈F^{k}(S^{k}). Remote data points (“outliers”) were not used for fitting the polynomials F. For each probe in the sample (element Sᵢ^{k} in the vector S^{k}), the normalized value (in log-space) Mᵢ^{k} is calculated from the initial value St by transforming it with the polynomial function F^{k}, so that Mᵢ^{k}=F^{k}(Sᵢ^{k}). Statistical analysis is performed in log-space. For presentation and calculation of fold-change, data is translated back to linear-space by taking the exponent.

f. qRT-PCR Assay

RNA was incubated in the presence of poly(A) polymerase (PAP; Takara-2180A), MnCl₂, and ATP for 1 h at 37° C. Then, using an oligodT primer harboring a consensus sequence, reverse transcription was performed on total RNA using SuperScript II RT (Invitrogen). Next the cDNA was amplified by real time PCR; this reaction contained a microRNA-specific forward primer, a TaqMan probe complementary to the 3′ of the specific microRNA sequence as well as to part of the polyA adaptor sequence, and a universal reverse primer complementary to the consensus 3′ sequence of the oligodT tail.

g. Data Analysis

In order to identify microRNA signatures that can be used to predict bladder cancer progression, the expression levels of microRNA in samples from invasive tumors, stable non muscle invasive tumors that did not progress (NP) and unstable non muscle invasive tumors that progressed (IP) were compared (see Table 1). P-values were calculated using a two-sided unpaired t-test on the log-transformed normalized signal, and significance level was adjusted using Benjamini and Hochberg's False Discovery Rate. Fold-changes were calculated by the change in the median values of the normalized fluorescence signal for each microRNA. For each microRNA, the ability to separate the two groups by the Receiver operating characteristic (ROC) curve was characterized and the calculated area under the ROC curve was marked as AUC. An optimal classifier which reaches sensitivity and specificity of 100% has AUC=1; a random classifier has AUC=0.5. To test the ability of microRNA expression levels to differentiate the NP from the IP non muscle invasive bladder tumors, an automatic classifier was constructed that chooses the three microRNAs with the highest AUC, and classifies using a nearest-neighbor classifier (KNN with K=1) in the space of these microRNAs (in log-space). The performance of this classifier was evaluated by leave-one-out cross validation (LOOCV) on the dataset that included the progressing (IP) and non-progressing (NP) non muscle invasive bladder tumors. In each round one sample is left out, the microRNAs are chosen; the classifier is trained on the remaining samples, and then used to classify the left out sample. This classifier reached sensitivity of 89% and specificity of 92%. Similar results were obtained using 10-fold cross validation in the same manner, or using simple SVM (linear kernel) or LDA classifiers.

### Example 2

### Specific microRNAs are Able to Predict the Risk of Invasiveness of Bladder Cancer

73 bladder tumors were removed using a transurethral resection procedure. 29 of these samples were classified as invasive and 44 were classified as non muscle invasive. Out of the 44 patients with non muscle invasive bladder cancer, 26 did not progress during the 5-year follow up, and 18 had a progression of tumor stage during the 5-year follow up. The first group was termed ° stable non muscle invasive′ (no-progression) and the second group was termed ‘unstable non muscle invasive’ (invasive progression). The microRNA expression levels of these samples were profiled by microarray and compared between the three groups. The main goal was to find microRNAs that are differentially expressed between the stable non muscle invasive tumors and the unstable non muscle invasive tumors (which progress to invasion), in order to predict progression in patients with non muscle invasive bladder cancer.

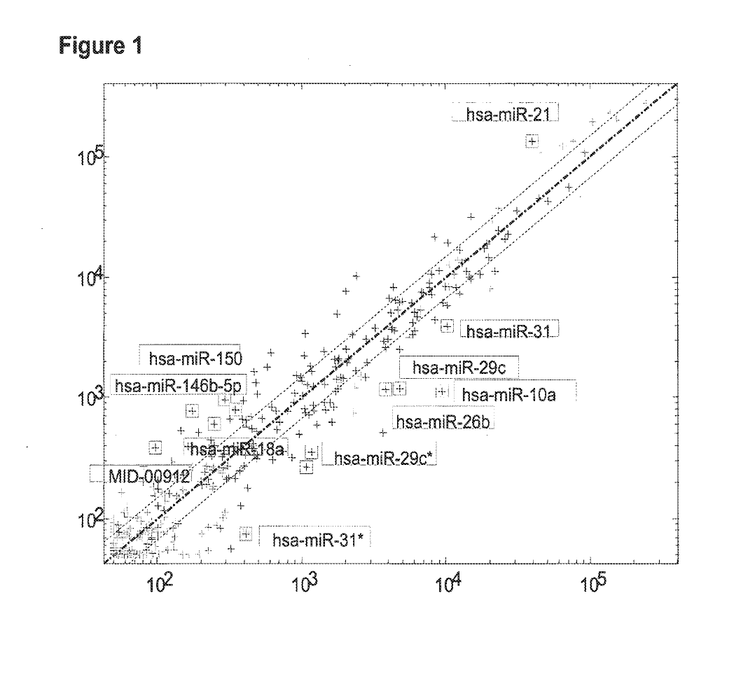

microRNA expression levels were first compared between the stable non muscle invasive samples and the invasive samples (FIG. 1). These are the two groups with the largest difference in the tumor characteristics, and therefore it was expected that if differences in microRNA expression within bladder cancer samples exist, they would be most pronounced between these two groups. As indicated in table 1, the microRNA expression profiles of these two groups were indeed significantly different, with 81 miRs differentially expressed (fold change of median expression above 1.2 and p-value which passed False Detection Rate of 0.05).

TABLE 1
Comparison of microRNA expression levels of invasive, stable non muscle
invasive (no progression to invasiveness) and unstable non muscle invasive (invasive
progression) tumors

| microR | | | | | | | | |
|---|---|---|---|---|---|---|---|---|
| NA | | | | | | | | |
| (the | | | | | | | | |
| miRBase | | hair | Invasive vs. | | Unstable vs. | | Invasive vs. | |
| registry | miR | pin | stable non | | stable non | | unstable non | |
| name, | SEQ | SEQ | muscle invasive | | muscle invasive | | muscle invasive | |
| release | ID | ID | p- | fold | p- | fold | p- | fold |
| 10) | NO: | NO: | value | change | value | change | value | change |
| hsa- | 8 | 24 | 5.90E−11 | 3.3 (−) | 3.00E−06 | 2.1 (−) | 3.40E−03 | 1.6 (−) |
| miR-26b | | | | | | | | |
| hsa- | 31 | 48, 49 | 1.50E−10 | 3.8 (+) | 3.90E−03 | 1.6 (+) | 1.30E−03 | 2.2 (+) |
| miR- | | | | | | | | |
| 199a-5p | | | | | | | | |
| hsa- | 2 | 17 | 2.00E−09 | 4.4 (+) | 1.30E−04 | 2.4 (+) | 3.10E−02 | 1.8 (+) |
| miR- | | | | | | | | |
| 146b-5p | | | | | | | | |
| hsa- | 6 | 21 | 2.10E−09 | 3.6 (+) | 9.20E−03 | 2.5 (+) | 5.30E−03 | 1.5 (+) |
| miR-575 | | | | | | | | |
| hsa- | 9 | 25 | 5.20E−08 | 4.0 (−) | 1.20E−04 | 2.1 (−) | 1.40E−02 | 1.8 (−) |
| miR-29c | | | | | | | | |
| hsa- | 15 | 25 | 7.40E−08 | 3.3 (−) | 7.80E−05 | 2.0 (−) | 9.90E−02 | 1.8 (−) |
| miR- | | | | | | | | |
| 29c* | | | | | | | | |
| hsa- | 1 | 16 | 1.30E−07 | 3.4 (+) | 7.30E−05 | 2.2 (+) | 4.20E−02 | 1.8 (+) |
| miR-21 | | | | | | | | |
| hsa- | 32 | 50 | 1.70E−07 | 1.9 (−) | 1.10E−04 | 1.5 (−) | 6.70E−02 | 1.3 (−) |
| miR- | | | | | | | | |
| 768-5p | | | | | | | | |
| hsa- | 33 | 51, 52 | 1.90E−07 | 4.2 (+) | 2.10E−03 | 1.6 (+) | 5.60E−03 | 2.4 (+) |
| miR- | | | | | | | | |
| 125b | | | | | | | | |
| hsa- | 34 | 53 | 2.50E−07 | 2.1 (+) | 1.80E−04 | 1.8 (+) | 4.00E−01 | 1.1 (+) |
| miR- | | | | | | | | |
| 130a | | | | | | | | |
| MID- | 35 | 54 | 6.90E−07 | 2.6 (−) | 5.40E−05 | 1.8 (−) | 1.20E−01 | 1.4 (−) |
| 00713# | | | | | | | | |
| MID- | 4 | 19 | 8.20E−07 | 4.0 (+) | 1.30E−03 | 3.1 (+) | 1.20E−01 | 1.3 (+) |
| 00912# | | | | | | | | |
| hsa- | 43 | 62 | 1.10E−06 | 3.8 (+) | 4.60E−03 | 1.6 (+) | 3.90E−02 | 2.3 (+) |
| miR-99a | | | | | | | | |
| hsa- | 13 | 28 | 1.50E−06 | 5.8 (−) | 1.00E−02 | 2.2 (−) | 1.80E−02 | 2.7 (−) |
| miR- | | | | | | | | |
| 29b-2* | | | | | | | | |
| hsa- | 10 | 26 | 1.60E−06 | 8.4 (−) | 4.30E−04 | 5.0 (−) | 2.80E−01 | 1.8 (−) |
| miR-10a | | | | | | | | |
| MID- | 36 | 55 | 1.00E−05 | 3.0 (−) | 1.20E−03 | 2.0 (−) | 4.90E−01 | 1.6 (−) |
| 00394# | | | | | | | | |
| hsa- | 37 | 56 | 1.40E−05 | 2.7 (−) | 9.10E−03 | 1.7 (−) | 9.50E−02 | 1.6 (−) |
| miR-98 | | | | | | | | |
| hsa- | 38 | 57 | 2.30E−05 | 1.8 (−) | 1.50E−03 | 1.6 (−) | 3.70E−01 | 1.1 (−) |
| miR-34a | | | | | | | | |
| hsa- | 7 | 22, 23 | 2.90E−05 | 4.1 (−) | 2.30E−06 | 5.2 (−) | 4.40E−01 | 1.2 (+) |
| miR-138 | | | | | | | | |
| hsa- | 39 | 58, 59 | 4.80E−05 | 1.7 (−) | 1.30E−04 | 1.8 (−) | 4.20E−01 | 1.0 (−) |
| miR-29b | | | | | | | | |
| hsa- | 40 | 60 | 5.80E−05 | 1.9 (−) | 6.80E−03 | 1.6 (−) | 1.60E−01 | 1.3 (−) |
| miR- | | | | | | | | |
| 768-3p | | | | | | | | |
| hsa- | 3 | 18 | 1.40E−04 | 2.4 (+) | 5.90E−04 | 2.1 (+) | 8.20E−01 | 1.1 (+) |
| miR-18a | | | | | | | | |
| hsa- | 14 | 29 | 3.10E−04 | 2.3 (+) | 2.00E−04 | 2.0 (+) | 8.70E−01 | 1.0 (+) |
| miR- | | | | | | | | |
| 193a-3p | | | | | | | | |
| hsa- | 41 | 16 | 3.80E−04 | 2.1 (+) | 2.10E−04 | 1.6 (+) | 7.10E−01 | 1.2 (+) |
| miR-21* | | | | | | | | |
| hsa- | 42 | 61 | 5.40E−04 | 1.7 (+) | 1.50E−05 | 1.9 (+) | 3.20E−01 | 1.1 (−) |
| miR-25 | | | | | | | | |
| hsa- | 5 | 20 | 6.00E−04 | 3.2 (+) | 1.40E−03 | 2.5 (+) | 1.00E+00 | 1.4 (+) |
| miR-150 | | | | | | | | |
| hsa- | 11 | 27 | 1.10E−03 | 5.5 (−) | 1.70E−03 | 4.3 (−) | 7.90E−01 | 1.3 (−) |
| miR-31* | | | | | | | | |
| hsa- | 44 | 63 | 1.90E−03 | 1.7 (+) | 2.40E−03 | 1.9 (+) | 7.50E−01 | 1.1 (−) |
| miR- | | | | | | | | |
| 130b | | | | | | | | |
| hsa-let | 45 | 64 | 2.20E−03 | 1.9 (−) | 6.60E−03 | 1.6 (−) | 4.70E−01 | 1.2 (−) |
| 7e | | | | | | | | |
| hsa- | 46 | 65 | 2.90E−02 | 1.5 (−) | 8.10E−03 | 1.6 (−) | 5.20E−01 | 1.0 (+) |
| miR-612 | | | | | | | | |
| hsa- | 47 | 30 | 4.10E−02 | 1.4 (−) | 5.50E−03 | 1.7 (−) | 5.20E−01 | 1.1 (+) |
| miR-27a | | | | | | | | |
| hsa- | 12 | 27 | 6.00E−02 | 2.6 (−) | 3.90E−03 | 4.0 (−) | 1.00E−01 | 1.4 (+) |
| miR-31 | | | | | | | | |

#These miRs are not in the miRBase registry and were cloned at the Rosetta Genomics laboratory.
P-values (two-sided unpaired t-test) and fold changes (of median normalized fluorescence) for comparisons between the 3 groups of bladder tumor samples. The table shows microRNAs that passed FDR of 0.05 and had changes greater 1.5-fold in median expression levels in the comparison of unstable vs. stable non muscle invasive bladder cancer. “+” marks higher expression in first group (more aggressive cancer) and “−” marks lower expression in the second group.

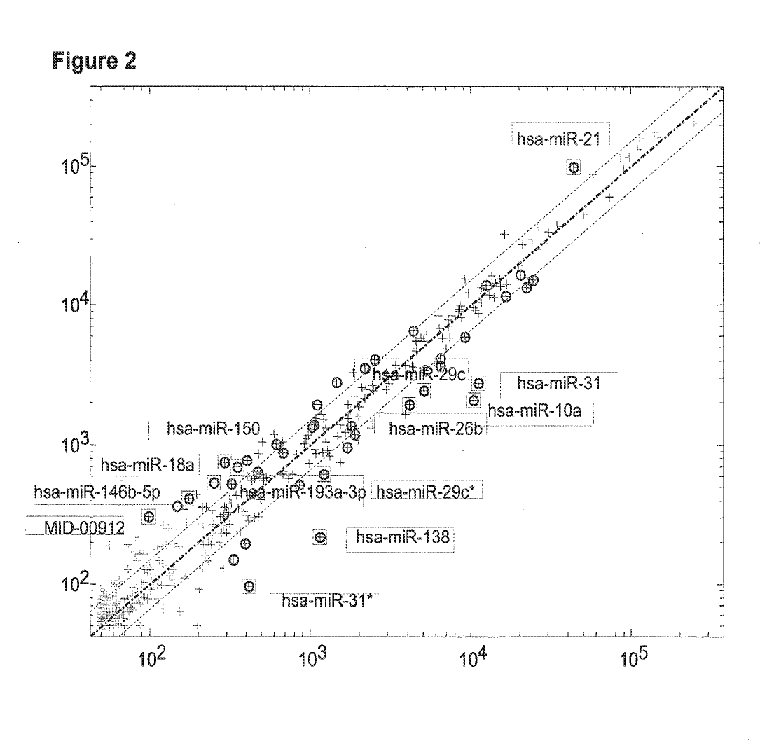

Next, the stable non muscle invasive bladder tumor samples were compared to the unstable non muscle invasive samples. Significant differences were found in the microRNA expression levels of the two groups (FIG. 2). 35 microRNAs (Table 1) had a fold change of median expression above 1.2 and passed False Discovery Rate (FDR) of 0.05 (p-value<0.013). Satisfyingly, 30 of the 35 microRNAs which were differentially expressed between stable non muscle invasive tumors and unstable non muscle invasive tumors were also differentially expressed between stable non muscle invasive tumors and invasive tumors (p-value<0.05, 29 are significant also at FDR=05) with an even stronger difference (higher fold changes and more significant p-values, Table 1). Furthermore, the microRNA expression profile of unstable non muscle invasive tumors had a high similarity to the microRNA expression profile of invasive tumors, much higher than its similarity to NP non muscle invasive tumors. Interestingly, even though the unstable non muscle invasive tumors and the invasive tumors differ in their stage, none of the microRNAs passed FDR of 0.05 when comparing the two groups. In comparison, 81 microRNAs were differentially expressed between the stable non muscle invasive tumors and the invasive tumors (at FDR=0.05 with fold-change above 1.2; data not shown). Thus, although histologically, non muscle invasive tumors differ from invasive tumors, the non muscle invasive tumors which will progress are already invasive-like on the molecular level.

For four of the patients, both a non muscle invasive tumor and an additional sample from a later invasive tumor were obtained. For each of these patients the two samples were compared. The correlation between pairs of samples from the same patient was very high (Pearson correlation coefficients between 0.95 and 0.96) relative to the correlation of random samples from the non muscle invasive group to random samples from the invasive group (mean Pearson correlation coefficient 0.87). This further supports the observation that a pattern of microRNA expression that is associated with tumor invasiveness is already present at the early non muscle invasive stage.

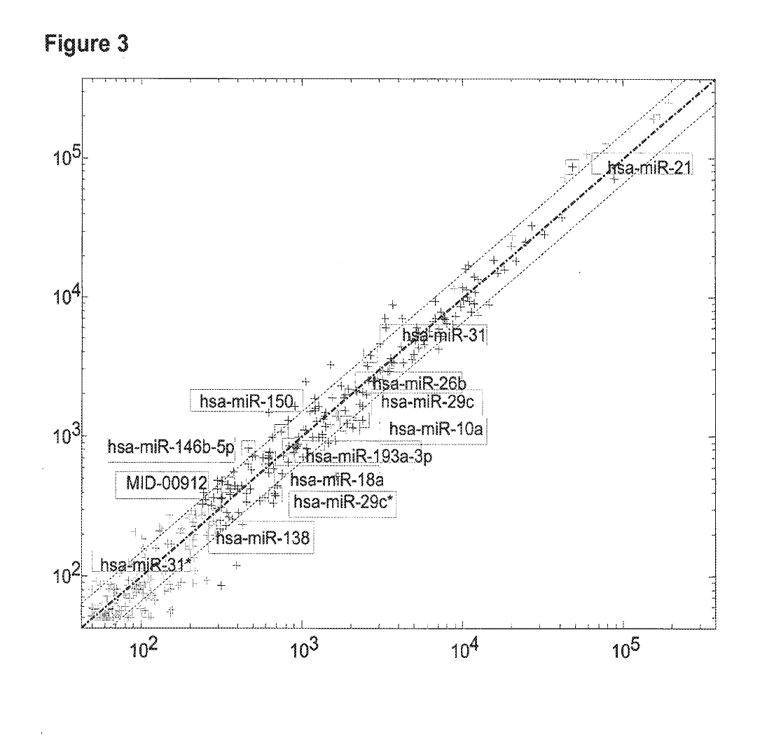

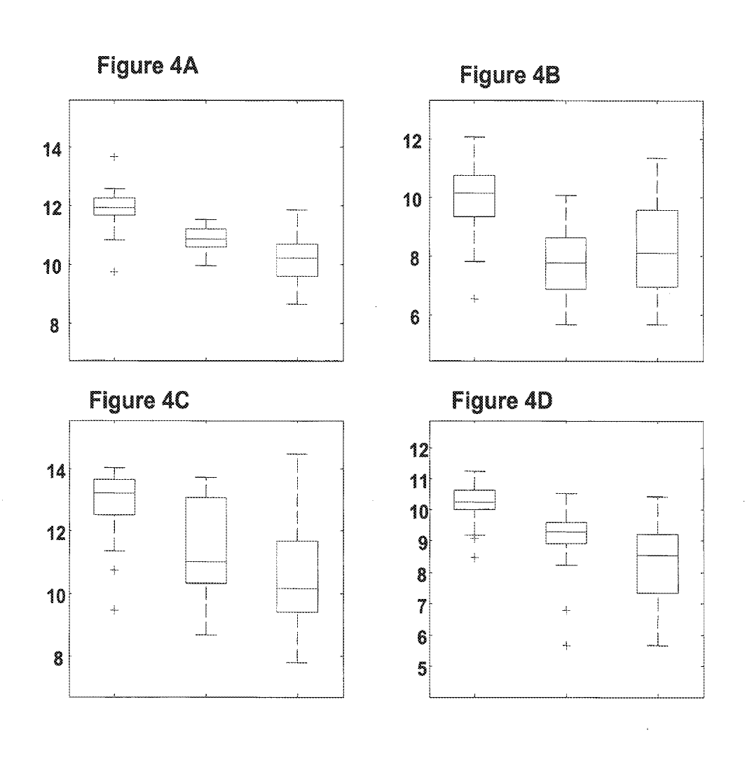

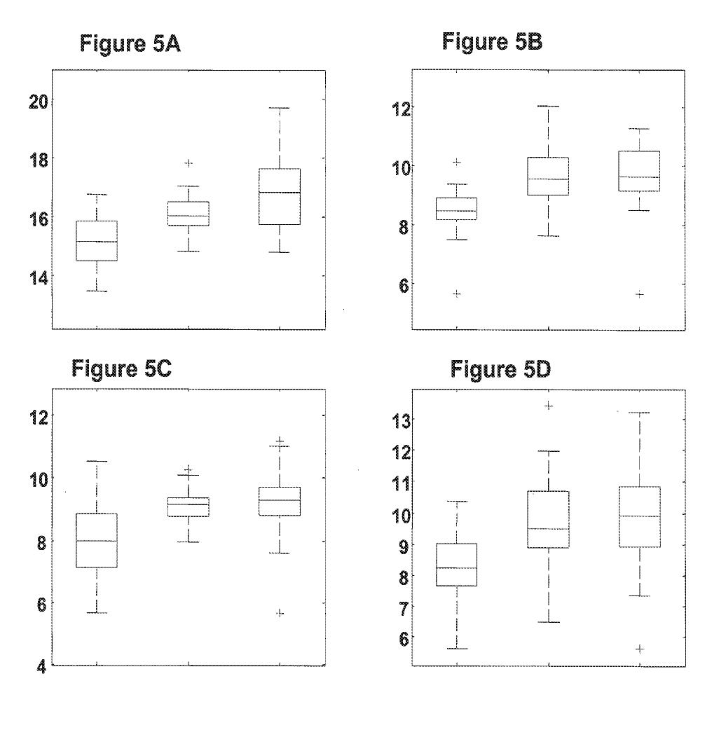

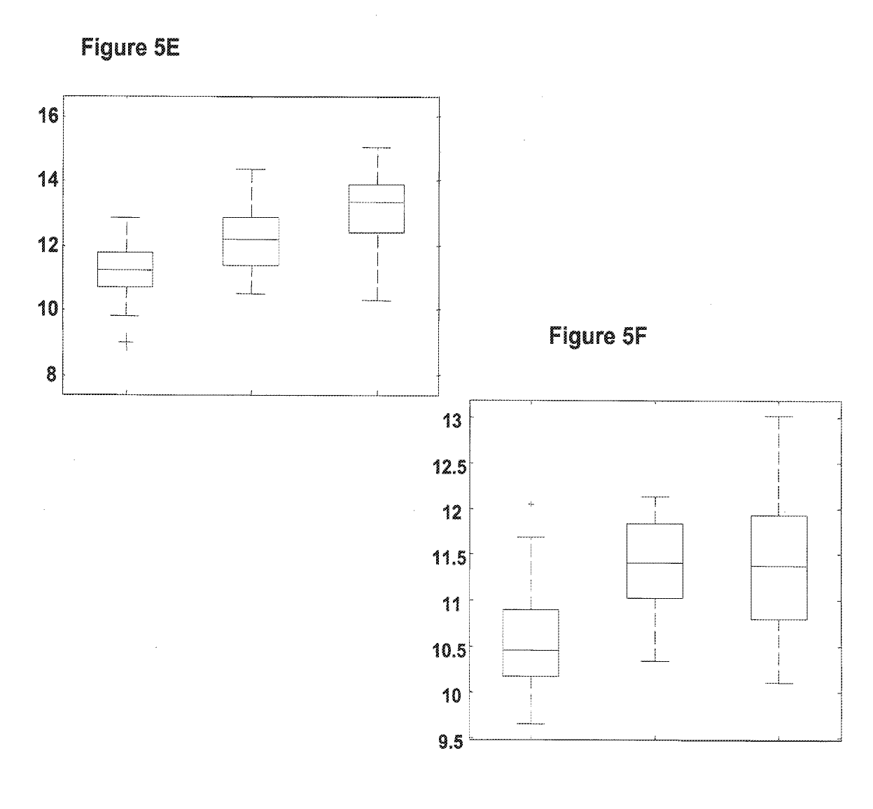

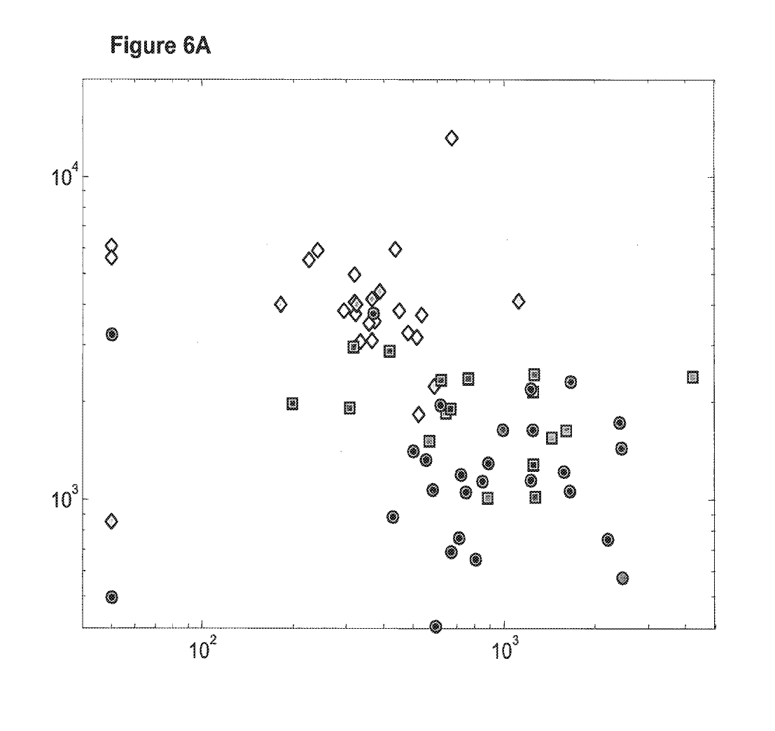

The statistical analysis of the microarray results and comparison of the median values of miRs expression in tumor samples obtained from bladder cancer patients with stable non muscle invasive tumor, unstable non muscle invasive tumor or invasive tumor, revealed a significant difference in the expression pattern of specific miRs as specified in Table 1. The normalized expression levels of hsa-miR-21 (SEQ ID NO: 1), hsa-miR-146b-5p (SEQ ID NO: 2), hsa-miR-18a (SEQ ID NO: 3), MID 00912 (SEQ ID NO: 4), hsa-miR-150 (SEQ ID NO: 5), hsa-miR-193a-3p (SEQ ID NO: 14) and hsa-miR-575 (SEQ ID NO: 6) were found to increase, while the normalized expression levels of hsa-miR-138 (SEQ ID NO: 7), hsa-miR-26b (SEQ ID NO: 8), hsa-miR-29c (SEQ ID NO: 9), hsa-miR-10a (SEQ ID NO: 10), miR-31* (SEQ ID NO: 11), hsa-miR-31 (SEQ ID NO: 12) hsa-miR-29c* (SEQ ID NO: 15) and hsa-miR-29b-2* (SEQ ID NO: 13) were found to decrease in tumor samples obtained from patients with unstable non muscle invasive tumor or from patients with invasive tumor as compared to tumor samples obtained from patients with stable non muscle invasive tumor (FIGS. 1-5). Accordingly, up regulation or down regulation of these miRs is demonstrated to be predictive of invasiveness of bladder cancer.

These miRs can be used to distinguish between stable non muscle invasive tumor and unstable non muscle invasive tumor. The classification could be conducted either with a simple threshold (1 or 2 dimension threshold), a logistic regression model or any other classifier.

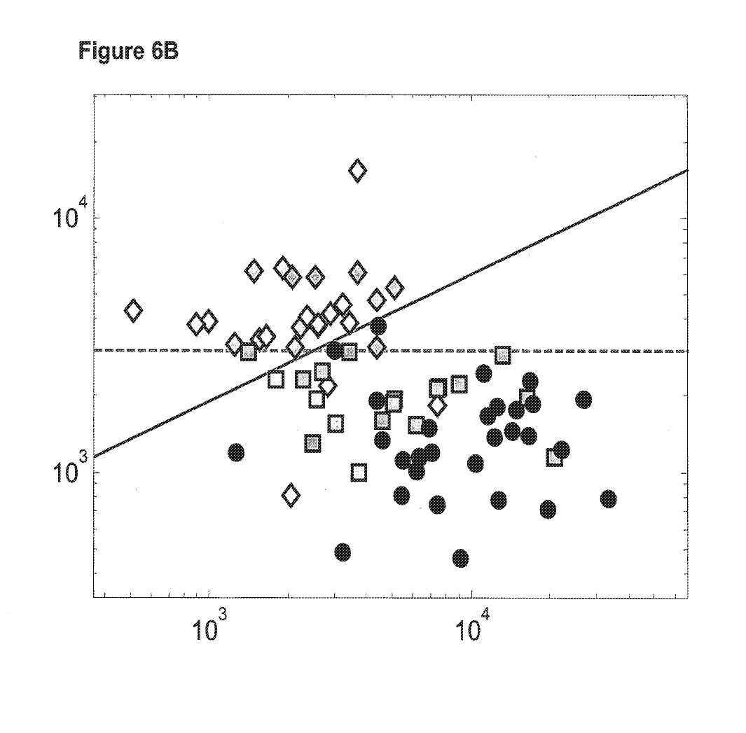

It was checked whether the differences in microRNA expression profiles of stable and unstable non muscle invasive samples could be used to predict progression. Using the expression levels of two microRNAs, hsa-miR-26b (SEQ ID NO: 8) and hsa-miR-193a-3p (SEQ ID NO: 14), that are differentially expressed between stable non muscle invasive and invasive tumors (Table 1), it is possible to identify non muscle invasive tumors that will progress to invasiveness within 5 years (unstable non muscle invasive tumors) with 76% sensitivity and 88% specificity (FIG. 6A). Accordingly, these microRNAs clearly separate invasive from stable non muscle invasive tumors, with unstable non muscle invasive tumors distributed with intermediate levels between these two groups. Additionally, the combination of these two miRs identified a subgroup (FIG. 6A, diagonal line) of tumors that should receive more aggressive treatments (sensitivity 80%, specificity 100%). When comparing only initially non muscle invasive tumors, a cutoff on hsa-miR-26 (SEQ ID NO: 8) alone identified those that progressed within 5 years with 100% sensitivity and 92% specificity.

A simple nearest neighbor classifier correctly classified 88% of the samples (85% spec and 94% sensitivity) in a leave-one-out cross-validation test.

An additional identification of subgroups of tumors is evident in FIG. 6B (diagonal line), which presents the combination of hsa-miR-26b (SEQ ID NO: 8) and hsa-miR-125b (SEQ ID NO: 33). The horizontal line in FIG. 6B shows that based on miR-26b (SEQ ID NO: 8) alone, the sensitivity of identifying unstable non muscle invasive tumors vs. stable non muscle invasive tumors is 100%, and the specificity is 88%.

Since tumor grade is used today to predict the risk of progression of non muscle invasive bladder cancer, the correlation between microRNA expression and tumor grade was checked in the cohort. microRNA expression levels of low grade (grade 1-2) versus high grade (grade 3) samples were compared within each of the two groups (stable and unstable non muscle invasive tumors) and no significant differences were found (no microRNAs passed a False Detection Rate as high as 0.4).

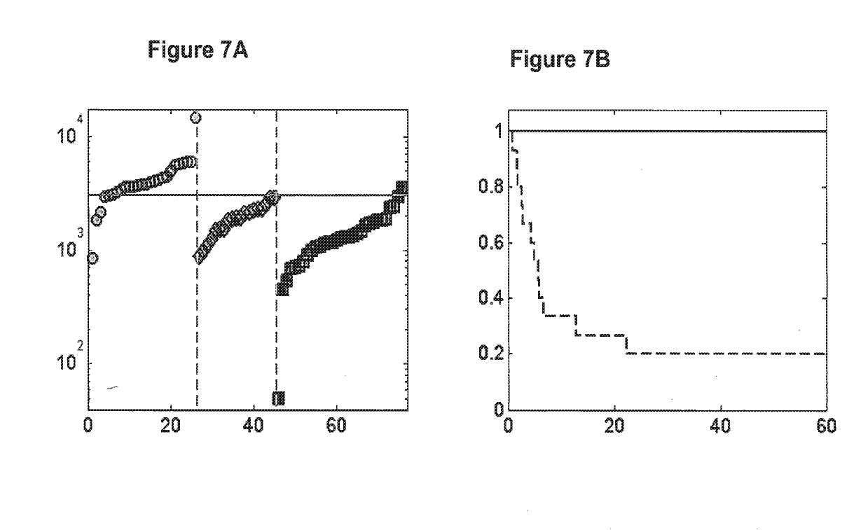

Using expression of hsa-miR-26b (SEQ ID NO: 8) to separate the non muscle invasive tumors into risk groups, a large and statistically significant difference in progression-free survival between these risk groups was obtained (FIGS. 7A-7B, p-value 3.1-E06).

Setting a classification threshold on the expression of hsa-miR-26b to 3020 (horizontal line in FIG. 7A), it is possible to distinguish unstable non muscle invasive tumors that will progress to invasiveness within 5 years from stable non muscle invasive tumors that will not progress with 100% sensitivity and 88% specificity (AUC=0.92, FIG. 7A). Indeed, separating the non muscle invasive tumors into risk groups based only on the expression of hsa-miR-26b, revealed a large and statistically significant difference in progression-free survival between these risk groups as evident from the Kaplan-Meier analysis (FIG. 7B). The 23 patients with non muscle invasive bladder tumors which had high expression of hsa-miR-26b (above threshold) had no cases of tumor progression whereas the 14 patients whose tumors had low expression of hsa-miR-26b had a median progression-free survival of only 5 months. The difference in progression-free survival was highly significant (p-value 4.3e-7 by log rank test). Thus, based on the expression of a single microRNA, one will be able to identify a high risk group of non muscle invasive bladder cancer patients with a positive predictive value for progression of 100%.

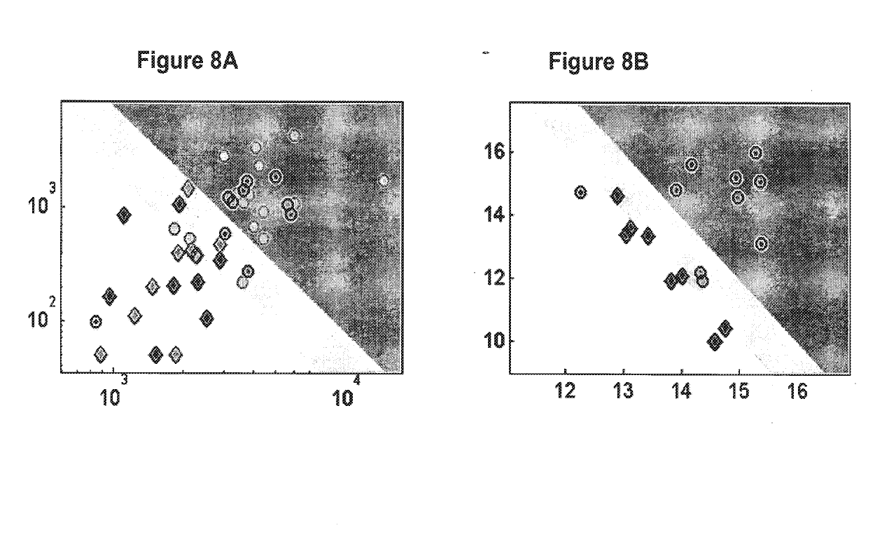

While hsa-miR-26b alone can be used to achieve a high classification rate on this data, using additional microRNAs will create a more robust margin. FIGS. 8A-8B show an example of a criterion for predicting whether a non muscle invasive tumor will become invasive. The classification rule is based on levels of hsa-miR-26b (SEQ ID NO: 8) and hsa-miR138 (SEQ ID NO: 7), and it has a 4 fold difference between scores of unstable non muscle invasive tumor vs. stable non muscle invasive tumor samples.

### Example 3 qRT-PCR Assay for Predicting the Risk of Invasiveness of Bladder Cancer

A qRT-PCR assay was performed, in accordance to example 1f. above, on a subset of the samples and microRNAs used in the microRNA assay described in Example 2.

Levels of five microRNAs which had different expression in unstable non muscle invasive vs. stable non muscle invasive samples were measured on nine of the unstable non muscle invasive samples and ten of the stable non muscle invasive samples. These miRs were hsa-miR-26b (SEQ ID NO: 8), hsa-miR-146b-5p (SEQ ID NO: 2), hsa-miR-21 (SEQ ID NO: 1), hsa-miR-25 (SEQ ID NO: 42) and hsa-miR-138 (SEQ ID NO: 7).

The sequences of the Fwd primers, MGB probes and reverse primer used in the PCR are provided in table 2 below.

TABLE 2
PCR primers and probes
Reverse primer: GCGAGCACAG AATTAATACG AC SEQ ID NO: 76

| | Fwd (Forward miR | SEQ ID | | SEQ ID |
|---|---|---|---|---|
| | specific) primer | NO: | MGB probe | NO: |
| hsa-miR-26b | CAGTCATTTGGCTT | 66 | CCGTTTTTTTTTTT | 71 |
| | CAAGTAATTCAGGA | | TACCTATCC | |
| hsa-miR-146b- | CAGTCATTTGGCTG | 67 | CCGTTTTTTTTTTT | 72 |
| 5p | AGAACTGAATTCCA | | TAGCCTATG | |
| hsa-miR-21 | CAGTCATTTGGCTA | 68 | CCGTTTTTTTTTTT | 73 |
| | GCTTATCAGACTGA | | TCAACATCA | |
| hsa-miR-25 | CAGTCATTTGGCCA | 69 | CCGTTTTTTTTTTT | 74 |
| | TTGCACTTGTCTCG | | TCAGACCGA | |
| hsa-miR-138 | CAGTCATTTGGCAG | 70 | CGTTTTTTTTTTTT | 75 |
| | CTGGTGTTGTGAAT | | CGGCCTGA | |

A comparison of the median expression of the miRs in stable non muscle invasive tumor samples vs. unstable non muscle invasive tumor samples, as found in the PCR assay, is presented in table 3.

TABLE 3
Median expression of miRs in stable non muscle invasive
tumor samples vs. unstable non muscle invasive tumor
samples, as found in PCR assay

| | | | Median | | | |
|---|---|---|---|---|---|---|
| | | | stable | Median | | |
| | | | non | unstable | | |
| | | SEQ | muscle | non muscle | | |
| | micro | ID | invasive | invasive | | Fold |
| | RNA | NO: | (50-Ct) | (50-Ct) | p-value | change |
| Up regulated | hsa- | 7 | 14.742 | 12.052 | 7.90E−03 | 6.45 |
| in stable vs. | miR- | | | | | |
| unstable non | 138 | | | | | |
| muscle | hsa- | 8 | 14.682 | 13.734 | 1.60E−01 | 1.93 |
| invasive | miR | | | | | |
| | 26b | | | | | |
| Down | hsa- | 2 | 12.715 | 13.708 | 1.50E−01 | 1.99 |
| regulated in | miR- | | | | | |
| stable vs. | 146b- | | | | | |
| unstable non | 5p | | | | | |
| muscle | hsa- | 42 | 17.826 | 18.571 | 3.80E−02 | 1.68 |
| invasive | miR- | | | | | |
| | 25 | | | | | |
| | hsa- | 1 | 20.769 | 21.388 | 1.30E−01 | 1.54 |
| | miR- | | | | | |
| | 21 | | | | | |

For the expression levels of all five microRNAs, the differences found in the PCR assay between the two groups of samples (Table 3) were similar in direction to the differences seen in the microarray results (Table 1). This similarity is also apparent in FIGS. 8A and 8B, which show expression results of the PCR assay and the microarray respectively, of hsa-miR-26b (SEQ ID NO: 8) and hsa-miR-138 (SEQ ID NO: 7) in bladder tumor samples obtained from patients with stable non muscle invasive tumor and in bladder tumor samples obtained from patients with unstable non muscle invasive tumor.

The foregoing description of the specific embodiments so fully reveals the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1. A method for determining a prognosis of bladder cancer in a human subject in need of treatment for bladder cancer, comprising:
(a) obtaining a biological sample from the subject;
(b) determining in said sample the expression level the nucleic acid sequences of SEQ ID NOS: 8-10, 15, 34 and 44 and sequences at least about 90% identical thereto; and
(c) comparing said obtained expression level to a threshold expression level, wherein an increase in the expression level of the nucleic acid sequences of SEQ ID NOS: 8-10 and is compared to said threshold expression level is indicative of a good prognosis and a decrease in the expression level of the nucleic acid sequence of SEQ ID NOS: 34 and 44 compared to said threshold expression level is indicative of poor prognosis for said subject; and
(d) determining the prognosis of bladder cancer in the subject based on the expression level of SEQ ID NOS: 8-10, 15, 34 and 44.

2. (canceled)

3. (canceled)

4. The method of claim 1, wherein said prognosis is prediction of bladder cancer risk of invasiveness.

5. The method of claim 1, wherein said altered expression level is a change in a score based on a polynomial combination of expression level of said nucleic acid sequence.

6. A method for distinguishing between stable non muscle invasive bladder cancer and unstable non muscle invasive bladder cancer in a subject in need of treatment for non-muscle invasive bladder cancer, said method comprising:
(a) obtaining a biological sample from a subject;
(b) determining in said sample an expression profile of nucleic acid sequences of SEQ ID NOS: 8-10, 15, 34 and 44, or a sequence having at least 90% identity thereto;
(c) comparing said expression profile to a reference value; whereby an increase in the expression level of the nucleic acid sequences of SEQ ID NOS: 8-10 and is compared to said reference value is indicative of stable non-muscle invasive bladder cancer and a decrease in the expression level of the nucleic acid sequences of SEQ ID NOS: 24 and 44 compared to said reference value is indicative of unstable non-muscle invasive bladder cancer; and
(d) distinguishing between stable and unstable non-muscle invasive bladder cancer in said subject based on the expression level of SEQ ID NOS: 8-10, 15, 34 and 44.

7.-9. (canceled)

10. The method of claim 1, wherein said biological sample is selected from the group consisting of bodily fluid, a cell line and a tissue sample.

11. The method of claim 10, wherein said tissue is a fresh, frozen, fixed, wax-embedded or formalin fixed paraffin-embedded (FFPE) tissue.

12. The method of claim 11, wherein said tissue is a bladder tissue.

13. (canceled)

14. The method of claim 1, wherein the expression level is determined by a method selected from the group consisting of nucleic acid hybridization, nucleic acid amplification, and a combination thereof.

15. The method of claim 14, wherein the nucleic acid hybridization is performed using a solid-phase nucleic acid biochip array or in situ hybridization.

16. The method of claim 14, wherein the nucleic acid amplification is performed using real-time PCR, said PCR method comprising forward and reverse primers, and further comprising a probe.

17. (canceled)

18. The method of claim 16, wherein the forward primers comprise a sequence selected from the group consisting of SEQ ID NOS: 66-70, a fragment thereof, and a sequence having at least about 80% identity thereto.

19. The method of claim 16, wherein the reverse primer comprises SEQ ID NO: 76, a fragment thereof, and a sequence having at least about 80% identity thereto.

20. (canceled)

21. The method of claim 16, wherein the probe comprises a sequence that is complementary to a sequence selected from the group consisting of SEQ ID NOS: 8-10, 15, 24 and 44, a fragment thereof, and a sequence having at least about 90% identity thereto.

22. The method of claim 16, wherein the probe comprises a sequence selected from the group consisting of SEQ ID NOS: 71-75, a fragment thereof, and a sequence having at least about 90% identity thereto.

23. A kit for determining a prognosis of a subject with bladder cancer, said kit comprising at least one probe comprising a nucleic acid sequence that is complementary to a sequence of SEQ ID NO: 8-10, 15, 34 or 44; to a fragment thereof or to a sequence at least about 80% identical thereto.

24. The kit of claim 23, wherein said probe comprises a nucleic acid sequence selected from SEQ ID NO: 71-75; to a fragment thereof or to a sequence at least about 80% identical thereto.

25. The kit of claim 23, wherein the kit further comprises forward and reverse primers.

26. The kit of claim 25, wherein the forward primers comprises a sequence selected from the group consisting of SEQ ID NOS: 66-70, a fragment thereof, and a sequence having at least about 80% identity thereto.

27. The kit of claim 25, wherein the reverse primer comprises SEQ ID NO: 76, a fragment thereof, and a sequence having at least about 80% identity thereto.

28. (canceled)


SEQUENCE LISTING (76 sequences)

SEQ ID NO: 1 - RNA, 22 nt, Homo Sapiens
uagcuuaucagacugauguuga

SEQ ID NO: 2 - RNA, 22 nt, Homo Sapiens
ugagaacugaauuccauaggcu

SEQ ID NO: 3 - RNA, 23 nt, Homo Sapiens
uaaggugcaucuagugcagauag

SEQ ID NO: 4 - RNA, 22 nt, Homo Sapiens
uggugcugcgggaacccagggu

SEQ ID NO: 5 - RNA, 22 nt, Homo Sapiens
ucucccaacccuuguaccagug

SEQ ID NO: 6 - RNA, 19 nt, Homo Sapiens
gagccaguuggacaggagc

SEQ ID NO: 7 - RNA, 23 nt, Homo Sapiens
agcugguguugugaaucaggccg

SEQ ID NO: 8 - RNA, 21 nt, Homo Sapiens
uucaaguaauucaggauaggu

SEQ ID NO: 9 - RNA, 22 nt, Homo Sapiens
uagcaccauuugaaaucgguua

SEQ ID NO: 10 - RNA, 23 nt, Homo Sapiens
uacccuguagauccgaauuugug

SEQ ID NO: 11 - RNA, 22 nt, Homo Sapiens
ugcuaugccaacauauugccau

SEQ ID NO: 12 - RNA, 21 nt, Homo Sapiens
aggcaagaugcuggcauagcu

SEQ ID NO: 13 - RNA, 22 nt, Homo Sapiens
cugguuucacaugguggcuuag

SEQ ID NO: 14 - RNA, 22 nt, Homo Sapiens
aacuggccuacaaagucccagu

SEQ ID NO: 15 - RNA, 22 nt, Homo Sapiens
ugaccgauuucuccugguguuc

SEQ ID NO: 16 - RNA, 72 nt, Homo Sapiens
ugucggguagcuuaucagacugauguugacuguugaaucucauggcaacaccagucgaugggcugucugaca

SEQ ID NO: 17 - RNA, 73 nt, Homo Sapiens
ccuggcacugagaacugaauuccauaggcugugagcucuagcaaugcccuguggacucaguucuggugcccgg

SEQ ID NO: 18 - RNA, 71 nt, Homo Sapiens
uguucuaaggugcaucuagugcagauagugaaguagauuagcaucuacugcccuaagugcuccuucuggca

SEQ ID NO: 19 - RNA, 82 nt, Homo Sapiens
gcuugagccaucccuggcuuccuggagcucacaaccugagagagaagguggugcugcgggaacccaggguugggcugugggc

SEQ ID NO: 20 - RNA, 84 nt, Homo Sapiens
cuccccauggcccugucucccaacccuuguaccagugcugggcucagacccugguacaggccugggggacagggaccuggggac

SEQ ID NO: 21 - RNA, 94 nt, Homo Sapiens
aauucagcccugccacuggcuuaugucaugaccuugggcuacucaggcugucugcacaaugagccaguuggacaggagcagugccacucaacuc

SEQ ID NO: 22 - RNA, 99 nt, Homo Sapiens
cccuggcauggugugguggggcagcugguguugugaaucaggccguugccaaucagagaacggcuacuucacaacaccagggccacaccacacuacagg

SEQ ID NO: 23 - RNA, 84 nt, Homo Sapiens
cguugcugcagcugguguugugaaucaggccgacgagcagcgcauccucuuacccggcuauuucacgacaccaggguugcauca

SEQ ID NO: 24 - RNA, 77 nt, Homo Sapiens
ccgggacccaguucaaguaauucaggauagguugugugcuguccagccuguucuccauuacuuggcucggggaccgg

SEQ ID NO: 25 - RNA, 88 nt, Homo Sapiens
aucucuuacacaggcugaccgauuucuccugguguucagagucuguuuuugucuagcaccauuugaaaucgguuaugauguaggggga

SEQ ID NO: 26 - RNA, 110 nt, Homo Sapiens
gaucugucugucuucuguauauacccuguagauccgaauuuguguaaggaauuuuguggucacaaauucguaucuaggggaauauguaguugacauaaacacuccgcucu

SEQ ID NO: 27 - RNA, 71 nt, Homo Sapiens
ggagaggaggcaagaugcuggcauagcuguugaacugggaaccugcuaugccaacauauugccaucuuucc

SEQ ID NO: 28 - RNA, 81 nt, Homo Sapiens
cuucuggaagcugguuucacaugguggcuuagauuuuuccaucuuuguaucuagcaccauuugaaaucaguguuuuaggag

SEQ ID NO: 29 - RNA, 88 nt, Homo Sapiens
cgaggaugggagcugagggcugggucuuugcgggcgagaugagggugucggaucaacuggccuacaaagucccaguucucggcccccg

SEQ ID NO: 30 - RNA, 78 nt, Homo Sapiens
cugaggagcagggcuuagcugcuugugagcaggguccacaccaagucguguucacaguggcuaaguuccgccccccag

SEQ ID NO: 31 - RNA, 23 nt, Homo Sapiens
cccaguguucagacuaccuguuc

SEQ ID NO: 32 - RNA, 26 nt, Homo Sapiens
guuggaggaugaaaguacggagugau

SEQ ID NO: 33 - RNA, 22 nt, Homo Sapiens
ucccugagacccuaacuuguga

SEQ ID NO: 34 - RNA, 22 nt, Homo Sapiens
cagugcaauguuaaaagggcau

SEQ ID NO: 35 - RNA, 22 nt, Homo Sapiens
uggugugcuagaguacucgaag

SEQ ID NO: 36 - RNA, 22 nt, Homo Sapiens
gagugugcuagaguccucgaag

SEQ ID NO: 37 - RNA, 22 nt, Homo Sapiens
ugagguaguaaguuguauuguu

SEQ ID NO: 38 - RNA, 22 nt, Homo Sapiens
uggcagugucuuagcugguugu

SEQ ID NO: 39 - RNA, 23 nt, Homo Sapiens
uagcaccauuugaaaucaguguu

SEQ ID NO: 40 - RNA, 28 nt, Homo Sapiens
ucacaaugcugacacucaaacugcugac

SEQ ID NO: 41 - RNA, 21 nt, Homo Sapiens
caacaccagucgaugggcugu

SEQ ID NO: 42 - RNA, 22 nt, Homo Sapiens
cauugcacuugucucggucuga

SEQ ID NO: 43 - RNA, 22 nt, Homo Sapiens
aacccguagauccgaucuugug

SEQ ID NO: 44 - RNA, 22 nt, Homo Sapiens
cagugcaaugaugaaagggcau

SEQ ID NO: 45 - RNA, 22 nt, Homo Sapiens
ugagguaggagguuguauaguu

SEQ ID NO: 46 - RNA, 25 nt, Homo Sapiens
gcugggcagggcuucugagcuccuu

SEQ ID NO: 47 - RNA, 21 nt, Homo Sapiens
uucacaguggcuaaguuccgc

SEQ ID NO: 48 - RNA, 71 nt, Homo Sapiens
gccaacccaguguucagacuaccuguucaggaggcucucaauguguacaguagucugcacauugguuaggc

SEQ ID NO: 49 - RNA, 110 nt, Homo Sapiens
aggaagcuucuggagauccugcuccgucgccccaguguucagacuaccuguucaggacaaugccguuguacaguagucugcacauugguuagacugggcaagggagagca

SEQ ID NO: 50 - RNA, 104 nt, Homo Sapiens
cugugcuuuguguguuggaggaugaaaguacggagugauccaucggcuaagugucuugucacaaugcugacacucaaacugcugacagcacacguuuuucacag

SEQ ID NO: 51 - RNA, 88 nt, Homo Sapiens
ugcgcuccucucagucccugagacccuaacuugugauguuuaccguuuaaauccacggguuaggcucuugggagcugcgagucgugcu

SEQ ID NO: 52 - RNA, 89 nt, Homo Sapiens
accagacuuuuccuagucccugagacccuaacuugugagguauuuuaguaacaucacaagucaggcucuugggaccuaggcggagggga

SEQ ID NO: 53 - RNA, 89 nt, Homo Sapiens
ugcugcuggccagagcucuuuucacauugugcuacugucugcaccugucacuagcagugcaauguuaaaagggcauuggccguguagug

SEQ ID NO: 54 - RNA, 66 nt, Homo Sapiens
cuuugccgagacuagagucacauccugacacaacucuuguccuggugugcuagaguacucgaagag

SEQ ID NO: 55 - RNA, 64 nt, Homo Sapiens
ucaucgaggcuagagucacgcuuggguaucggcuauugccugagugugcuagaguccucgaaga

SEQ ID NO: 56 - RNA, 119 nt, Homo Sapiens
aggauucugcucaugccagggugagguaguaaguuguauuguugugggguagggauauuaggccccaauuagaagauaacuauacaacuuacuacuuucccugguguguggcauauuca

SEQ ID NO: 57 - RNA, 110 nt, Homo Sapiens
ggccagcugugaguguuucuuuggcagugucuuagcugguuguugugagcaauaguaaggaagcaaucagcaaguauacugcccuagaagugcugcacguuguggggccc

SEQ ID NO: 58 - RNA, 81 nt, Homo Sapiens
cuucaggaagcugguuucauauggugguuuagauuuaaauagugauugucuagcaccauuugaaaucaguguucuuggggg

SEQ ID NO: 59 - RNA, 81 nt, Homo Sapiens; identical to SEQ ID NO: 28

SEQ ID NO: 60 - RNA, 104 nt, Homo Sapiens; identical to SEQ ID NO: 50

SEQ ID NO: 61 - RNA, 84 nt, Homo Sapiens
ggccaguguugagaggcggagacuugggcaauugcuggacgcugcccugggcauugcacuugucucggucugacagugccggcc

SEQ ID NO: 62 - RNA, 81 nt, Homo Sapiens
cccauuggcauaaacccguagauccgaucuuguggugaaguggaccgcacaagcucgcuucuaugggucugugucagugug

SEQ ID NO: 63 - RNA, 82 nt, Homo Sapiens
ggccugcccgacacucuuucccuguugcacuacuauaggccgcugggaagcagugcaaugaugaaagggcaucggucagguc

SEQ ID NO: 64 - RNA, 79 nt, Homo Sapiens
cccgggcugagguaggagguuguauaguugaggaggacacccaaggagaucacuauacggccuccuagcuuuccccagg

SEQ ID NO: 65 - RNA, 100 nt, Homo Sapiens
ucccaucuggacccugcugggcagggcuucugagcuccuuagcacuagcaggaggggcuccaggggcccucccuccauggcagccaggacaggacucuca

SEQ ID NO: 66 - DNA, 28 bp, Artificial Sequence
  Synthetic
cagtcatttggcttcaagtaattcagga

SEQ ID NO: 67 - DNA, 28 bp, Artificial Sequence
  Synthetic
cagtcatttggctgagaactgaattcca

SEQ ID NO: 68 - DNA, 28 bp, Artificial Sequence
  Synthetic
cagtcatttggctagcttatcagactga

SEQ ID NO: 69 - DNA, 28 bp, Artificial Sequence
  Synthetic
cagtcatttggccattgcacttgtctcg

SEQ ID NO: 70 - DNA, 28 bp, Artificial Sequence
  Synthetic
cagtcatttggcagctggtgttgtgaat

SEQ ID NO: 71 - DNA, 23 bp, Artificial Sequence
  Synthetic
ccgttttttttttttacctatcc

SEQ ID NO: 72 - DNA, 23 bp, Artificial Sequence
  Synthetic
ccgttttttttttttagcctatg

SEQ ID NO: 73 - DNA, 23 bp, Artificial Sequence
  Synthetic
ccgttttttttttttcaacatca

SEQ ID NO: 74 - DNA, 23 bp, Artificial Sequence
  Synthetic
ccgttttttttttttcagaccga

SEQ ID NO: 75 - DNA, 22 bp, Artificial Sequence
  Synthetic
cgttttttttttttcggcctga

SEQ ID NO: 76 - DNA, 22 bp, Artificial Sequence
  Synthetic
gcgagcacagaattaatacgac